(19) United States
(12) Patent Application Publication
(10) Pub. No.: US 2021/0213020 A1
(43) Pub. Date: Jul. 15, 2021

(54) METHODS AND PHARMACEUTICAL COMPOSITIONS FOR TREATING CANCER

(71) Applicants: INSERM (INSTITUT NATIONAL DE LA SANTÉ ET DE LA RECHIRCHE MÉDICALE), Paris (FR); UNIVERSITÉ PARIS-SACLAY, Saint Aubin (FR); ASSISTANCE PUBLIQUE-HÔPITAUS DE PARIS (APHP), Paris (FR)

(72) Inventor: David MACHOVER, PARIS (FR)

(21) Appl. No.: 17/059,379

(22) PCT Filed: May 29, 2019
(86) PCT No.: PCT/EP2019/063936

§ 371 Date: Nov. 27, 2020

(30) Foreign Application Priority Data
May 30, 2018 (EP) 18305661.3

(51) Int. Cl. A61K 31/519; A61K 31/4415; A61K 31/513; A61K 45/06

(52) U.S. Cl. CPC A61K 31/519; A61K 45/06; A61K 31/513; A61K 31/4415

(57) ABSTRACT

Disclosed are methods and pharmaceutical compositions for treating cancer. It was hypothesized that low activity of serine hydroxymethyl transferase (SHMT) due to poor pyridoxal 5′-phosphate (PLP) availability within cancer cells would result in insufficient growth inhibition in cancer cells exposed to 5-fluorouracil (FUra), as well as to FUra in combination with N5-formyl tetra hydro pteroylglutamate (5-HCO—H4PteGlu; folinic acid). Cancer cell lines were exposed to FUra as a single agent and to FUra with folinic acid, in combination with high concentration PLP. There was demonstrated synergistic and additive interactions upon cytotoxicity of FUra by folinic acid and PLP combined in HT29, HCT116, and L1210 cancer cells. Murine studies of parenteral administration of pyridoxamine or pyridoxine in high doses showed that intracellular PLP is augmented to levels close or greater than the Kd reported for binding of cofactor to SHMT, suggesting modulation of the fluoropyrimidines by vitamin B6 was possible.

### FIELD OF THE INVENTION

The present invention relates to methods and pharmaceutical compositions for treating cancer in a subject in need thereof.

### BACKGROUND OF THE INVENTION

Since its introduction in the 50ies by Heidelberg et al., the drug of choice for the treatment of metastatic colorectal carcinoma has been 5-fluorouracil (FUra), a fluorinated analog of uracil.

Biochemical studies demonstrated that the main route of FUra action proceeds via complex metabolic pathways that result in the formation of 5-fluorodeoxyuridine monophosphate (FdUMP), a potent inhibitor of thymidylate synthase (TS).

Mechanistically, it was demonstrated that FdUMP inactivates TS by forming a covalent ternary complex consisting of FdUMP, TS and 5,10-methylenetetrahydrofolate (CH₂—H₄PteGlu) with concomitant inhibition of the enzyme. Stability of the complex depends on concentration of CH₂—H₄PteGlu. Indeed, the rate of dissociation of FdUMP from the ternary complex decreases as the concentration of CH₂—H₄PteGlu is increased. Low concentrations of the cofactor lead to quick dissociation of the complex and rapid recovery of TS activity resulting in loss of cytotoxic activity.

It was thus proposed to potentiate the inhibition of TS by FUra by increasing intracellular levels of reduced folates, in particular by co-administering FUra and folinic acid (Leucovorin). However, the attainment of high intracellular levels of CH₂—H₄PteGlu remains an issue: neither under basal conditions of growth, nor after supplementation of tumor cells with folates in high doses, are the intracellular levels of CH₂—H₄PteGlu sufficiently high to allow optimal TS inhibition by FUra. Indeed, when cancer cells are exposed to high concentrations of reduced folates with the aim to expand the intracellular folate pools, increase of CH₂—H₄PteGlu levels is only small (Machover et al. (2001) Biochemical Pharmacol. 61:867-876), and rapid decrease occurs after discontinuation of folate exposure.

Accordingly, there is still an important need for improved antitumor combinations enabling optimal TS inhibition by FUra.

### SUMMARY OF THE INVENTION

The present invention relates to methods and pharmaceutical compositions for treating cancer in a subject in need thereof.

The present invention also relates to an antitumor pharmaceutical combination or composition comprising (i) a fluoropyrimidine, (ii) a B6 vitamer, and optionally (iii) a folate.

The present invention also relates to an antitumor pharmaceutical combination or composition comprising (i) a fluoropyrimidine, (ii) a B6 vitamer, and optionally (iii) a folate for use in the treatment of cancer in a subject in need thereof.

### DETAILED DESCRIPTION OF THE INVENTION

The inventors thought that low activity of serine hydroxymethyl transferase (SHMT) due to poor pyridoxal 5′-phosphate (PLP) availability within cancer cells would result in insufficient growth inhibition in cancer cells exposed to 5-fluorouracil (FUra), as well as to FUra in combination with N5-formyl tetra hydro pteroylglutamate (5-HCO—H4PteGlu; folinic acid). The present invention arises from the unexpected finding by the inventors that the cytotoxic activity of FUra, without and with reduced folates, was synergistically increased by addition of high doses of a B6 vitamer, namely pyridoxal 5′-phosphate (PLP), a 5′-phosphorylated derivative of pyridoxal. The inventors indeed demonstrated that additional potentiation of fluoropyrimidines could be achieved by increasing the availability of CH₂—H₄PteGlu through improvement of the PLP-dependent metabolic folate interconversion leading to the formation of CH₂—H₄PteGlu, by addition of high doses of PLP.

Cancer cell lines grown in vitro were exposed to FUra as a single agent and to FUra with folinic acid, in combination with high concentration PLP. The inventors demonstrated synergistic and additive interactions upon cytotoxicity of FUra by folinic acid and PLP combined in HT29, HCT116, and L1210 cancer cells. Murine studies of parenteral administration of pyridoxamine in high doses showed that intracellular PLP is augmented to levels close or greater than the Kd reported for binding of cofactor to SHMT, which suggests that modulation of the fluoropyrimidines by vitamin B6 could be achieved in vivo.

Accordingly, the present invention relates to an antitumor pharmaceutical combination comprising (i) a fluoropyrimidine, (ii) a B6 vitamer, and optionally (iii) a folate.

The present invention also relates to this antitumor pharmaceutical combination for simultaneous, separate or sequential use in the treatment of cancer in a subject in need thereof.

Another object of the invention concerns an antitumor pharmaceutical composition comprising (i) a fluoropyrimidine, (ii) a B6 vitamer, and optionally (iii) a folate.

The present invention also concerns this antitumor pharmaceutical composition for use, optionally in combination with a folate, in the treatment of cancer in a subject in need thereof.

Another object of the invention concerns a fluoropyrimidine for use in combination with a B6 vitamer and optionally a folate in the treatment of cancer in a subject in need thereof.

Another object of the invention concerns a B6 vitamer for use in combination with a fluoropyrimidine and optionally a folate in the treatment of cancer in a subject in need thereof.

Another object of the invention concerns a folate for use in combination with a fluoropyrimidine and a B6 vitamer in the treatment of cancer in a subject in need thereof. The present invention further concerns a kit comprising:

a) (i) an antitumor pharmaceutical composition comprising a fluoropyrimidine and a B6 vitamer and (ii) one or more dosage units of a folate, or

b) (i) an antitumor pharmaceutical composition comprising a fluoropyrimidine and (ii) one or more dosage units of a pharmaceutical composition comprising a B6 vitamer and optionally a folate, or

c) (i) an antitumor pharmaceutical composition comprising a fluoropyrimidine, (ii) one or more dosage units of a B6 vitamer and (iii) one or more dosage units of a folate.

The present invention further concerns a combined preparation comprising (i) one or more dosage units of a fluoropyrimidine, (ii) one or more dosage units of a B6 vitamer, and optionally (iii) one or more dosage units of a folate, for use in the treatment of cancer in a subject in need thereof.

### Fluoropyrimidine

As used herein the terms “fluoropyrimidine” or “fluoropyrimidine compound” refer to fluorinated pyrimidines which have antitumor activity through several mechanisms including inhibition of RNA synthesis and function, inhibition of thymidylate synthase activity and altered DNA synthesis. Examples of fluoropyrimidines include 5-fluorouracil (FUra), capecitabine (a prodrug of doxifluridine), 5-fluoro-2′-deoxyuridine (FUdR), ftorafur (a prodrug of FUra), emitefur (a combination of the FUra prodrug 1-ethoxymethyl FUra and the dihydropyrimidine dehydrogenase inhibitor 3-cyano-2,6-dihydroxypyridine in a 1:1 molar ratio), a combination of eniluracil (an uracil analogue which inhibits dihydropyrimidine dehydrogenase) and FUra, S-1 (a combination of ftorafur and two FUra modulators called 5-chloro-2,4-dihydroxypuridine and oxonic acid in a 1:0.4:1 molar ratio), UFT (a combination of ftorafur and uracil in a 1:4 molar ratio), any fluoropyrimidine whose active metabolite is fluorodeoxyuridine monophosphate (FdUMP), and mixtures thereof.

Particularly, the fluoropyrimidine used in the context of the invention is selected from the group consisting of 5-fluorouracil (FUra), capecitabine, 5-fluoro-2′-deoxyuridine (FUdR), ftorafur, emitefur, eniluracil/5-FU, S-1, UFT, any fluoropyrimidine whose active metabolite is fluorodeoxyuridine monophosphate (FdUMP) and mixtures thereof. Particularly, the fluoropyrimidine used in the context of the invention is selected from the group consisting of 5-fluorouracil (FUra), capecitabine, 5-fluoro-2′-deoxyuridine (FUdR), ftorafur, emitefur, eniluracil/5-FU, S-1, UFT and mixtures thereof. Particularly, the fluoropyrimidine used in the context of the invention is 5-fluorouracil.

The dosing and administration techniques for fluoropyrimidines are well-known in the art, and their optimization for a specific patient is within the ability of the skilled clinician.

The fluoropyrimidines used in the context of the invention may be administered by oral or parenteral route, in particular by oral or intravenous route. When administered by intravenous route, the fluoropyrimidines may be used in the context of the invention by bolus administration, short-duration intravenous infusion, continuous infusion or a mixture thereof. Typically, bolus dosing with FUra may be administered to a subject in a dosage amount of from 370 to 500 mg/m2 daily for 5 days every 3 to 5 weeks, or 500 mg/m2 weekly. Alternatively, one or more doses of FUra may be given by continuous infusion over a period of at least 22 hours per dose. Continuous dosing with FUra may include an intravenous bolus dose of 400 mg/m2 followed by 600 mg/m2 over 22 hours for two days. Alternatively, a bolus dose of 400 mg/m2 of FUra may be followed by a dose of 2400 mg/m2 administered over 46 hours. A number of other different schedules of FUra well-known from the skilled person can also be used in the context of the present invention.

### B6 Vitamer

As used herein, the term “B6 vitamer” refers to any compound or mixture of compounds that has any biological activity in any biological assay for vitamin B6. B6 vitamers include, but are not limited to, pyridoxine (also called pyridoxol or PN), pyridoxal (PL), pyridoxamine (PM), the 5′ phosphorylated derivatives of any of the three aforementioned compounds, namely pyridoxine 5′-phosphate (PNP), pyridoxamine 5′-phosphate (PMP) and pyridoxal 5′-phosphate (PLP), acetate esters thereof, pharmaceutically acceptable salts thereof and any derivative or related compound that can be converted to PLP, PNP or PMP in a test organism. Thus, for example, the acetate esters or other esters of any of the available hydroxyl groups of any of the aforementioned six compounds, and which are likely to be hydrolyzed by specific or non-specific esterases, are included in B6 vitamers. Also, various salts, such as hydrochloride salts, of any of the aforementioned compounds are included in B6 vitamers.

Particularly, the B6 vitamer used in the context of the invention is selected from the group consisting of pyridoxine (PN), pyridoxal (PL), pyridoxamine (PM), 5′-phosphorylated derivatives thereof, acetate esters thereof, pharmaceutically compatible salts thereof, and mixtures thereof.

The B6 vitamers used in the context of the invention may be administered by oral or parenteral route, in particular by oral, intravenous or intramuscular route. When administered by intravenous route, the B6 vitamers may be used in the context of the invention by bolus administration, continuous infusion (preferably from a few hours to several days, more preferably from one hour to 5 days) or a mixture thereof.

Particularly, the B6 vitamers used in the context of the invention are administered at a high dose.

By “administering B6 vitamers at a high dose” is meant herein administering B6 vitamers at a higher dose than the one conventionally used to treat B6 vitamin deficiency. Doses of B6 vitamers conventionally used to treat B6 vitamin deficiency are well-known from the skilled person. Typically, conventional doses of B6 vitamers used to treat B6 vitamin deficiency are 1.3-300 mg/day, more particularly 50-300 mg/day.

Particularly, the B6 vitamers used in the context of the invention are administered at a dose at least twice higher, particularly at least three times higher, at least four time higher, at least five times higher, at least six times higher, at least seven times higher, at least eight times higher, at least nine times higher, at least ten times higher, at least twenty times higher, at least thirty times higher, at least forty times higher or at least fifty times higher than the dose conventionally used to treat B6 vitamin deficiency. Still higher doses of B6 vitamers may also be used in the context of the invention. Particularly, the B6 vitamers used in the context of the invention (e.g., PN, PL, PM or any of their 5′-phosphorylated derivatives) are administered at a high dose enabling to achieve plasma and/or intracellular levels of PLP equal or greater than that required for optimum synergistic effect of the fluoropyrimidines, typically equal or higher than 160 μmon.

### Folate

As used herein, the term “folate” refers to folic acid (pteroylglutamate), one or more of the pteroylglutamate compounds in which the pyrazine ring of the pterin moiety is reduced to give dihydrofolates or tetrahydofolates, or derivatives of all the preceding compounds in which the N-5 or the N-5 and N-10 positions carry one carbon units at various levels of oxidation, or pharmaceutically compatible salt thereof or a combination of two or more thereof.

Particularly, the folate used in the context of the invention is selected from the group consisting of folic acid, dihydrofolate, tetrahydrofolate, 5-methyltetrahydrofolate, 5,10-methylenetetrahydrofolate, 5,10-methenyltetrahydrofolate, 5-formiminotetrahydrofolate, 5-formyltetrahydrofolate (leucovorin or folinic acid), [6S]-5-formyltetrahydrofolate, 10-formyltetrahydrofolate, pharmaceutically compatible salts thereof, and mixtures thereof. Most particularly, the folate used in the context of the invention is 5-formyltetrahydrofolate or [6S]-5-formyltetrahydrofolate.

Within all folates or derivatives thereof, both the natural and the unnatural diastereoisomers, pharmaceutically compatible salts thereof and any mixtures of the isomers and the salts, but especially the natural diastereoisomeric forms such as [6S]-5-methyl tetrahydrofolic acid, [6R]-5,10-methylene tetrahydrofolic acid, and [6S]-5-formyl tetrahydrofolic acid are applicable.

The dosing and administration techniques for folates in the treatment of tumor diseases are well-known in the art, and their optimization for a specific patient is within the ability of the skilled clinician.

The folates used in the context of the invention may be administered by oral or parenteral route, in particular by oral, intravenous, intramuscular or subcutaneous route. When administered by intravenous route, the folates may be used in the context of the invention by bolus administration, continuous infusion or a mixture thereof. Typically, the folates, in particular folinic acid, may administered in a dosage amount of from 20 to 1000 mg/m²/day, more particularly in a dosage amount from 25 to 500 mg/m2/day, particularly in a dosage amount of from 50 to 400 mg/m2/day, still particularly in a dosage amount of from 100 to 200 mg/m2/day. In a particular embodiment, the folates, in particular folinic acid, may be administered at a low dose, i.e. at a dose ≤25 mg/m2/day. Alternatively, the folates, in particular folinic acid, may be administered at a high dose, i.e. at a dose ≥200 mg/m2/day. Particularly, the folates, in particular folinic acid, may be administered at a high dose to allow a therapeutically effective plasmatic concentration, i.e. a plasmatic concentration of 10 μM.

### Antitumor Pharmaceutical Combination

As used herein, the term “combination”, “therapeutic combination” or “pharmaceutical combination”, defines either a fixed combination in one dosage unit form or a kit of parts for the combined administration where the fluropyrimidine and the B6 vitamer (and optionally the folate) may be administered independently at the same time or separately within time intervals that allow that the combination partners show a synergistic effect.

The compounds of the combination of the invention can thus be formulated in one, two, three or more separate pharmaceutical compositions.

The present invention thus concerns an antitumor pharmaceutical composition comprising (i) a fluoropyrimidine as defined in the section “Fluoropyrimidines” above and (ii) a B6 vitamer as defined in the section “B6 vitamer” above. The present invention also concerns an antitumor pharmaceutical composition comprising (i) a fluoropyrimidine as defined in the section “Fluoropyrimidines” above, (ii) a B6 vitamer as defined in the section “B6 vitamer” above and (iii) a folate as defined in the section “Folates” above.

The present invention also concerns a kit comprising:

a) (i) an antitumor pharmaceutical composition comprising a fluoropyrimidine as defined in the section “Fluoropyrimidines” above, and a B6 vitamer, as defined in the section “B6 vitamer” above, and (ii) one or more dosage units of a folate as defined in the section “Folates” above, or

b) (i) an antitumor pharmaceutical composition comprising a fluoropyrimidine as defined in the section “Fluoropyrimidines” above and (ii) one or more dosage units of a pharmaceutical composition comprising a B6 vitamer as defined in the section “B6-vitamer” and optionally a folate as defined in the section “Folates” above, or

c) (i) an antitumor pharmaceutical composition comprising a fluoropyrimidine as defined in the section “Fluoropyrimidines” above, (ii) one or more dosage units of a B6 vitamer as defined in the section “B6 vitamer” above and (iii) one or more dosage units of a folate as defined in the section “Folates” above.

The kits according to the present invention thus comprise at least the two or three separate compositions (i), (ii) and (iii) defined above.

The term “pharmaceutical composition” is defined herein to refer to a mixture or solution containing at least one therapeutic agent to be administered to a subject, e.g., a mammal or human, in order to prevent or treat a particular disease or condition affecting the mammal.

Typically, the compounds of the combination of the invention may thus be combined with pharmaceutically acceptable excipients to form pharmaceutical compositions. The pharmaceutical compositions defined herein thus particularly further comprise pharmaceutically acceptable excipients.

“Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, and intranasal administration forms and rectal administration forms.

The compounds of the combination of the invention can be formulated in one, two, three or more separate pharmaceutical compositions, each composition being for the same or different administration routes.

Appropriate administration routes for each compound of the combination of the invention are well-known from the skilled person. For example, the fluoropyrimidine, as defined in the section “Fluoropyrimidine” above may be administered orally, transdermally or parenterally, in particular intravenously, intraperitoneally or by intraarterial route. Accordingly, the fluoropyrimidine, as defined in the section “Fluoropyrimidine” above can be formulated in a pharmaceutical composition for oral, transdermal or parenteral, such as intravenous, intraarterial or intraperitoneal, administration. The B6 vitamer, as defined in the section “B6 vitamer” above may be administered orally or parenterally, in particular intravenously or intramuscularly. Accordingly, the B6 vitamer, as defined in the section “B6 vitamer” above can be formulated in a pharmaceutical composition for oral or parenteral, such as intravenous or intramuscular, administration. The folate, as defined in the section “Folates” above may be administered orally or parenterally, in particular intravenously, subcutaneously or intramuscularly. Accordingly, the folate, as defined in the section “Folates” above can be formulated in a pharmaceutical composition for oral or parenteral, such as intravenous, subcutaneous or intramuscular, administration.

In a particular embodiment of the invention, the fluoropyrimidine as defined in the section “Fluoropyrimidine” above and the B6 vitamer as defined in the section “B6 vitamer” above are formulated in a single pharmaceutical composition, particularly in a single pharmaceutical composition for oral or parenteral, such as intravenous, administration. In another particular embodiment of the invention, the fluoropyrimidine as defined in the section “Fluoropyrimidine” above, the B6 vitamer as defined in the section “B6 vitamer” above and the folate as defined in the section “Folates” above are formulated in a single pharmaceutical composition for oral or parenteral, such as intravenous, administration.

In another particular embodiment of the invention, the fluoropyrimidine as defined in the section “Fluoropyrimidine” above and the B6 vitamer as defined in the section “B6 vitamer” above are formulated in a single pharmaceutical composition, particularly in a single pharmaceutical composition for oral or parenteral, such as intravenous, administration, and the folate as defined in the section “Folates” above is formulated in a separate pharmaceutical composition, particularly a pharmaceutical composition for oral or parenteral, such as intravenous, subcutaneous or intramuscular, administration.

In still another particular embodiment of the invention, the fluoropyrimidine as defined in the section “Fluoropyrimidine” above and the B6 vitamer as defined in the section “B6 vitamer” above are formulated in separate pharmaceutical compositions, the fluoropyrimidine being particularly formulated in a pharmaceutical composition for oral, transdermal or parenteral, such as intravenous, intraarterial or intraperitoneal, administration, and the B6 vitamer being particularly formulated in a separate pharmaceutical composition for oral or parenteral, such as intravenous or intramuscular, administration.

In another particular embodiment, the fluoropyrimidine as defined in the section “Fluoropyrimidine” above, the B6 vitamer as defined in the section “B6 vitamer” above and the folate as defined in the section “Folates” above are formulated in separate pharmaceutical compositions, the fluoropyrimidine being particularly formulated in a pharmaceutical composition for oral, transdermal or parenteral, such as intravenous, intraarterial or intraperitoneal, administration, the B6 vitamer being particularly formulated in a separate pharmaceutical composition for oral or parenteral, such as intravenous or intramuscular, administration, and the folate being particularly formulated in another separate pharmaceutical composition for oral or parenteral, such as intravenous, subcutaneous or intramuscular, administration.

In still another particular embodiment of the invention, the B6 vitamer as defined in the section “B6 vitamer” above and the folate as defined in the section “Folates” above are formulated in a single pharmaceutical composition, particularly in a single pharmaceutical composition for oral or parenteral, such as intravenous or intramuscular, administration, and the fluoropyrimidine as defined in the section “Fluoropyrimidines” above is formulated in a separate pharmaceutical composition, particularly a pharmaceutical composition for oral, transdermal or parenteral, such as intravenous, intraarterial or intraperitoneal, administration.

Particularly, in particular when the pharmaceutical compositions are for parenteral administration, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions can be prepared by incorporating the active compound in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The preparation of more, or highly concentrated solutions for direct injection is also contemplated, where the use of DMSO as solvent is envisioned to result in extremely rapid penetration, delivering high concentrations of the active agents to a small tumor area.

### Treatment of Cancer

The present invention concerns the antitumor pharmaceutical combination as defined in the section “Antitumor pharmaceutical combination” above for simultaneous, separate or sequential use in the treatment of cancer in a subject in need thereof.

Another object of the invention relates to a method for treating cancer in a subject comprising the simultaneous, separate or sequential administration of a therapeutically effective amount of the antitumor pharmaceutical combination, defined in the section “Antitumor pharmaceutical combination” above, in a subject in need thereof.

A further object of the invention concerns the use of (i) a fluoropyrimidine as defined in the section “Fluoropyrimidine” above, (ii) a B6 vitamer as defined in the section “B6 vitamer” above, and optionally (iii) a folate as defined in the section “Folate” above, in the manufacture of a antitumor pharmaceutical combined preparation for simultaneous, separate or sequential administration for the treatment of cancer.

The present invention also concerns a combined preparation comprising (i) one or more dosage units of a fluoropyrimidine as defined in the section “Fluoropyrimidine” above, (ii) one or more dosage units of a B6 vitamer as defined in the section “B6 vitamer” above, and optionally (iii) one or more dosage units of a folate as defined in the section “Folates” above, for use in the treatment of cancer.

The present invention further concerns an antitumor pharmaceutical composition comprising (i) a fluoropyrimidine, as defined in the section “Fluoropyrimidine” above and (ii) a B6 vitamer, as defined in the section “B6 vitamer” above, for use, optionally in combination with a folate, as defined in the section “Folate” above, in the treatment of cancer.

Another object of the invention relates to a method for treating cancer in a subject in need thereof comprising the step of administering a therapeutically effective amount of an antitumor pharmaceutical composition comprising (i) a fluoropyrimidine, as defined in the section “Fluoropyrimidine” above and (ii) a B6 vitamer, as defined in the section “B6 vitamer” above, and optionally the simultaneous, separate or sequential administration of a therapeutically effective amount of a folate, as defined in the section “Folate” above, in a subject in need thereof.

A further object of the invention concerns the use of (i) a fluoropyrimidine as defined in the section “Fluoropyrimidine” above and (ii) a B6 vitamer as defined in the section “B6 vitamer” above, in the manufacture of a medicament for the treatment of a tumor disease, said medicament being optionally within an antitumor pharmaceutical combined preparation including a folate, as defined in the section “Folate” above, for simultaneous, separate or sequential administration for the treatment of cancer.

The term “a combined preparation” is defined herein to refer to especially a “kit of parts” in the sense that the combination partners (i) and (ii) and optionally (iii), as defined above, can be dosed independently or by use of different fixed combinations with distinguished amounts of the combination partners, i.e., simultaneously or at different time points. The parts of the kit of parts can then e.g., be administered simultaneously or chronologically staggered, that is at different time points and with equal or different time intervals for any part of the kit of parts. The ratio of the total amounts of the combination partner (i) to the combination partner (ii) (and if applicable to the combination partner (iii)) to be administered in the combined preparation can be varied, e.g., in order to cope with the needs of a patient sub-population to be treated or the needs of the single patient.

The term “co-administration” or “combined administration” as used herein is defined to encompass the administration of the selected therapeutic agents to a single patient, and are intended to include treatment regimens in which the agents are not necessarily administered by the same route of administration or at the same time.

In a particular embodiment of the invention, the fluoropyrimidine as defined in the section “Fluoropyrimidine” above and the B6 vitamer as defined in the section “B6 vitamer” above are administered simultaneously, particularly orally or parenterally, in particular intravenously, in the form of a single pharmaceutical composition. In that embodiment, a folate as defined in the section “Folates” above may be administered orally or parenterally, in particular intravenously, intramuscularly or subcutaneously, in a separate pharmaceutical composition, simultaneously or immediately prior to the administration of the fluoropyrimidine and the B6 vitamer.

In another embodiment of the invention, the fluoropyrimidine as defined in the section “Fluoropyrimidine” above is administered orally, transdermally or parenterally, in particular intravenously, intraarterially or intraperitoneally, and the B6 vitamer is administered orally or parenterally, in particular intravenously or intramuscularly, in a separate pharmaceutical composition, simultaneously, prior or after the administration of the fluoropyrimidine. In that embodiment, a folate, as defined in the section “Folates” above may be administered orally or parenterally, in particular intravenously or intramuscularly, in the same pharmaceutical composition as the B6 vitamer. Alternatively, the folate may be administered orally or parenterally, in particular intravenously, intramuscularly or subcutaneously, in a separate pharmaceutical composition, simultaneously or immediately prior to the administration of the fluoropyrimidine and/or the B6 vitamer.

As used herein, the term “subject” denotes a mammal. Typically, a subject according to the invention refers to any subject (preferably human) afflicted or at risk to be afflicted with cancer. In a particular embodiment, the term “subject” refers to a subject afflicted or at risk to be afflicted with colorectal cancer. In a particular embodiment, the term “subject” refers to a subject afflicted or at risk to be afflicted with lymphocytic leukemia.

In the context of the invention, the term “treating” or “treatment” means reversing, alleviating, inhibiting the progress of, or preventing the disorder or condition to which such term applies, or one or more symptoms of such disorder or condition. As used herein, the term “treatment” or “treat” refer to both prophylactic or preventive treatment as well as curative or disease modifying treatment, including treatment of subjects at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

By a “therapeutically effective amount” of a compound of the invention is meant a sufficient amount of the compound to treat a cancer, (for example, to limit growth or to slow or block tumor metastasis) at a reasonable benefit/risk ratio applicable to any medical treatment. It will be understood, however, that the total daily usage of the compounds of the present invention will be decided by the attending physician within the scope of sound medical judgment. The specific therapeutically effective dose level for any particular subject will depend upon a variety of factors including the disorder being treating and the severity of the disorder, activity of the specific compounds employed, the specific combinations employed, the age, body weight, general health, sex and diet of the subject, the time of administration, route of administration and rate of excretion of the specific compounds employed, the duration of the treatment, drugs used in combination or coincidental with the specific compounds employed, and like factors well known in the medical arts. For example, it is well within the skill of the art to start doses of the compounds at levels lower than those required to achieve the desired therapeutic effect and to gradually increase the dosage until the desired effect is achieved.

The antitumor pharmaceutical combination or composition according to the invention is particularly useful to treat tumor diseases.

It should in particular be noted that the combination of the invention is useful to treat tumor diseases as such, and not side effects induced by the administration of one of the components of the combination, such as the hand-foot syndrome or palmar-plantar erythrodysesthesia.

The antitumor pharmaceutical combination or composition of the invention can be used to treat a perceptible tumor in any phase of its development, including a tumor disease in its advanced phase of evolution, or to treat imperceptible minimal residual tumor diseases. In particular, the antitumor pharmaceutical combination of the invention can used to treat small size primary tumor disease and metastatic cancer disease, including micrometastasis and disseminated tumor diseases.

More particularly, the term “treatment of cancer” or “treatment of a tumor disease” as used herein includes at least one of the following features: alleviation of the symptoms associated with the tumorous disease, a reduction in the extent of the tumorous disease (e.g. a reduction in tumor growth), a stabilization of the state of the tumorous disease (e.g. an inhibition of tumor growth), a prevention of further spread of the tumorous disease (e.g. a metastasis), a prevention of the occurrence or recurrence of a tumorous disease, a delaying or retardation of the progression of the tumorous disease (e.g. a reduction in tumor growth) or an improvement in the state of the tumorous disease (e.g. a reduction in tumor size).

The term “cancer” as used herein refers to “tumor disease” and includes every local increase in tissue volume as well as cells in which normal growth regulation no longer operates and uncontrolled cell division takes place. Examples of tumor diseases which can be treated with the aid of the antitumor pharmaceutical combination or composition of the invention include every tumor disease for the treatment of which fluoropyrimidines showed some effectiveness.

As used herein, the term “cancer” has its general meaning in the art and includes, but is not limited to, solid tumors and blood borne tumors. The term cancer includes diseases of the skin, tissues, organs, bone, cartilage, blood and vessels. The term “cancer” further encompasses both primary and metastatic cancers. Examples of cancers that may be treated by methods and compositions of the present invention include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic, lymphocytic leukemia, chronic lymphocytic leukemia; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the subject suffers from a cancer selected from the group consisting of colorectal cancer, prostate cancer, pancreatic cancer, colon cancer, rectal cancer, breast cancer, lung cancer, testicular cancer, brain cancer, skin cancer, gastric cancer, esophageal cancer, gastroesophageal cancer, biliary tract cancer, sarcomas, tracheal cancer, head and neck cancer, liver cancer, ovarian cancer, lymphoid cancer, cervical cancer, vulvar cancer, melanoma, mesothelioma, kidney cancer, renal cancer, urogenital cancers, bladder cancer, thyroid cancer, bone cancers, carcinomas, sarcomas, squamous-cell cancer, and soft tissue cancers.

In some embodiments, the subject suffers from cancer resistant to anti-cancer treatment. In some embodiments, the combination or composition of the present invention is administered sequentially or concomitantly with one or more therapeutic active agent such as to anti-cancer compound, chemotherapeutic or radiotherapeutic agents.

The term “anti-cancer compound” has its general meaning in the art and refers to anti-cancer compounds used in anti-cancer therapy such as tyrosine kinase inhibitors, tyrosine kinase receptor (TKR) inhibitors, EGFR tyrosine kinase inhibitors, anti-EGFR compounds, anti-HER2 compounds, Vascular Endothelial Growth Factor Receptors (VEGFRs) pathway inhibitors, interferon therapy, alkylating agents, anti-metabolites, immunotherapeutic agents, Interferons (IFNs), Interleukins, and chemotherapeutic agents such as described below.

The term “tyrosine kinase inhibitor” or “TKI” has its general meaning in the art and refers to any of a variety of therapeutic agents or drugs such as compounds inhibiting tyrosine kinase, tyrosine kinase receptor inhibitors (TKRI), EGFR tyrosine kinase inhibitors, EGFR antagonists. The term “tyrosine kinase inhibitor” or “TKI” has its general meaning in the art and refers to any of a variety of therapeutic agents or drugs that act as selective or non-selective inhibitors of receptor and/or non-receptor tyrosine kinases. Tyrosine kinase inhibitors and related compounds are well known in the art and described in U.S Patent Publication 2007/0254295, which is incorporated by reference herein in its entirety. It will be appreciated by one of skill in the art that a compound related to a tyrosine kinase inhibitor will recapitulate the effect of the tyrosine kinase inhibitor, e.g., the related compound will act on a different member of the tyrosine kinase signaling pathway to produce the same effect as would a tyrosine kinase inhibitor of that tyrosine kinase. Examples of tyrosine kinase inhibitors and related compounds suitable for use in methods of embodiments of the present invention include, but are not limited to Erlotinib, sunitinib (Sutent; SU11248), dasatinib (BMS-354825), PP2, BEZ235, saracatinib, gefitinib (Iressa), erlotinib (Tarceva; OSI-1774), lapatinib (GW572016; GW2016), canertinib (CI 1033), semaxinib (SU5416), vatalanib (PTK787/ZK222584), sorafenib (BAY 43-9006), imatinib (Gleevec; ST1571), leflunomide (SU101), vandetanib (Zactima; ZD6474), MK-2206 (8-[4-aminocyclobutyl)phenyl]-9-phenyl-1,2,4-triazolo [3,4-f][1,6]naphthyridin-3(2H)-one hydrochloride) derivatives thereof, analogs thereof, and combinations thereof. Additional tyrosine kinase inhibitors and related compounds suitable for use in the present invention are described in, for example, U.S Patent Publication 2007/0254295, U.S. Pat. Nos. 5,618,829, 5,639,757, 5,728,868, 5,804,396, 6,100,254, 6,127,374, 6,245,759, 6,306,874, 6,313,138, 6,316,444, 6,329,380, 6,344,459, 6,420,382, 6,479,512, 6,498,165, 6,544,988, 6,562,818, 6,586,423, 6,586,424, 6,740,665, 6,794,393, 6,875,767, 6,927,293, and 6,958,340, all of which are incorporated by reference herein in their entirety. In certain embodiments, the tyrosine kinase inhibitor is a small molecule kinase inhibitor that has been orally administered and that has been the subject of at least one Phase I clinical trial, more preferably at least one Phase II clinical, even more preferably at least one Phase III clinical trial, and most preferably approved by the FDA for at least one hematological or oncological indication. Examples of such inhibitors include, but are not limited to Erlotinib, Gefitinib, Lapatinib, Canertinib, BMS-599626 (AC-480), Neratinib, KRN-633, CEP-11981, Imatinib, Nilotinib, Dasatinib, AZM-475271, CP-724714, TAK-165, Sunitinib, Vatalanib, CP-547632, Vandetanib, Bosutinib, Lestaurtinib, Tandutinib, Midostaurin, Enzastaurin, AEE-788, Pazopanib, Axitinib, Motasenib, OSI-930, Cediranib, KRN-951, Dovitinib, Seliciclib, SNS-032, PD-0332991, MKC-I (Ro-317453; R-440), Sorafenib, ABT-869, Brivanib (BMS-582664), SU-14813, Telatinib, SU-6668, (TSU-68), L-21649, MLN-8054, AEW-541, and PD-0325901.

EGFR tyrosine kinase inhibitors as used herein include, but are not limited to compounds selected from the group consisting of but not limited to Erlotinib, lapatinib, Rociletinib (CO-1686), gefitinib, Dacomitinib (PF-00299804), Afatanib, Brigatinib (AP26113), WJTOG3405, NEJ002, AZD9291, HM61713, EGF816, ASP 8273, AC 0010. Examples of antibody EGFR inhibitors include Cetuximab, panitumumab, matuzumab, zalutumumab, nimotuzumab, necitumumab, Imgatuzumab (GA201, R05083945), and ABT-806.

In some embodiments, the combination or composition of the present invention is administered with a chemotherapeutic agent. The term “chemotherapeutic agent” refers to chemical compounds that are effective in inhibiting tumor growth. Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaorarnide and trimethylolomelamine; acetogenins (especially bullatacin and bullatacinone); a carnptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estrarnustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin (11 and calicheamicin 211, see, e.g., Agnew Chem Intl. Ed. Engl. 33:183-186 (1994); dynemicin, including dynemicin A; an esperamicin; as well as neocarzinostatin chromophore and related chromoprotein enediyne antiobiotic chromomophores), aclacinomysins, actinomycin, authramycin, azaserine, bleomycins, cactinomycin, carabicin, canninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idanrbicin, marcellomycin, mitomycins, mycophenolic acid, nogalarnycin, olivomycins, peplomycin, potfiromycin, puromycin, quelamycin, rodorubicin, streptomgrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophospharnide glycoside; aminolevulinic acid; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; podophyllinic acid; 2-ethylhydrazide; procarbazine; PSK®; razoxane; rhizoxin; sizofiran; spirogennanium; tenuazonic acid; triaziquone; 2,2′,2″-trichlorotriethylarnine; trichothecenes (especially T-2 toxin, verracurin A, roridinA and anguidine); urethan; vindesine; dacarbazine; mannomustine; mitobromtol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.].) and doxetaxel (TAXOTERE®, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-1 1; topoisomerase inhibitor RFS 2000; difluoromethylornithine (DMFO); retinoic acid; capecitabine; and pharmaceutically acceptable salts, acids or derivatives of any of the above. Also included in this definition are antihormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

In some embodiments, the combination or composition of the present invention is administered with a targeted cancer therapy. Targeted cancer therapies are drugs or other substances that block the growth and spread of cancer by interfering with specific molecules (“molecular targets”) that are involved in the growth, progression, and spread of cancer. Targeted cancer therapies are sometimes called “molecularly targeted drugs”, “molecularly targeted therapies”, “precision medicines”, or similar names. In some embodiments, the targeted therapy consists of administering the subject with a tyrosine kinase inhibitor as defined above.

In some embodiments, combination or composition of the present invention is administered with an immunotherapeutic agent. The term “immunotherapeutic agent,” as used herein, refers to a compound, composition or treatment that indirectly or directly enhances, stimulates or increases the body's immune response against cancer cells and/or that decreases the side effects of other anticancer therapies. Immunotherapy is thus a therapy that directly or indirectly stimulates or enhances the immune system's responses to cancer cells and/or lessens the side effects that may have been caused by other anti-cancer agents. Immunotherapy is also referred to in the art as immunologic therapy, biological therapy biological response modifier therapy and biotherapy. Examples of common immunotherapeutic agents known in the art include, but are not limited to, immune checkpoint inhibitor, cytokines, cancer vaccines, monoclonal antibodies and non-cytokine adjuvants. Alternatively the immunotherapeutic treatment may consist of administering the subject with an amount of immune cells (T cells, NK, cells, dendritic cells, B cells . . . ). Immunotherapeutic agents can be non-specific, i.e. boost the immune system generally so that the human body becomes more effective in fighting the growth and/or spread of cancer cells, or they can be specific, i.e. targeted to the cancer cells themselves immunotherapy regimens may combine the use of non-specific and specific immunotherapeutic agents. Non-specific immunotherapeutic agents are substances that stimulate or indirectly improve the immune system. Non-specific immunotherapeutic agents have been used alone as a main therapy for the treatment of cancer, as well as in addition to a main therapy, in which case the non-specific immunotherapeutic agent functions as an adjuvant to enhance the effectiveness of other therapies (e.g. cancer vaccines). Non-specific immunotherapeutic agents can also function in this latter context to reduce the side effects of other therapies, for example, bone marrow suppression induced by certain chemotherapeutic agents. Non-specific immunotherapeutic agents can act on key immune system cells and cause secondary responses, such as increased production of cytokines and immunoglobulins. Alternatively, the agents can themselves comprise cytokines. Non-specific immunotherapeutic agents are generally classified as cytokines or non-cytokine adjuvants. A number of cytokines have found application in the treatment of cancer either as general non-specific immunotherapies designed to boost the immune system, or as adjuvants provided with other therapies. Suitable cytokines include, but are not limited to, interferons, interleukins and colony-stimulating factors. Interferons (IFNs) contemplated by the present invention include the common types of IFNs, IFN-alpha (IFN-α), IFN-beta (IFN-β) and IFN-gamma (IFN-γ). IFNs can act directly on cancer cells, for example, by slowing their growth, promoting their development into cells with more normal behaviour and/or increasing their production of antigens thus making the cancer cells easier for the immune system to recognise and destroy. IFNs can also act indirectly on cancer cells, for example, by slowing down angiogenesis, boosting the immune system and/or stimulating natural killer (NK) cells, T cells and macrophages. Recombinant IFN-alpha is available commercially as Roferon (Roche Pharmaceuticals) and Intron A (Schering Corporation). Interleukins contemplated by the present invention include IL-2, IL-4, IL-11 and IL-12. Examples of commercially available recombinant interleukins include Proleukin® (IL-2; Chiron Corporation) and Neumega® (IL-12; Wyeth Pharmaceuticals). Zymogenetics, Inc. (Seattle, Wash.) is currently testing a recombinant form of IL-21, which is also contemplated for use in the combinations of the present invention. Colony-stimulating factors (CSFs) contemplated by the present invention include granulocyte colony stimulating factor (G-CSF or filgrastim), granulocyte-macrophage colony stimulating factor (GM-CSF or sargramostim) and erythropoietin (epoetin alfa, darbepoietin). Treatment with one or more growth factors can help to stimulate the generation of new blood cells in subjects undergoing traditional chemotherapy. Accordingly, treatment with CSFs can be helpful in decreasing the side effects associated with chemotherapy and can allow for higher doses of chemotherapeutic agents to be used. Various-recombinant colony stimulating factors are available commercially, for example, Neupogen® (G-CSF; Amgen), Neulasta (pelfilgrastim; Amgen), Leukine (GM-CSF; Berlex), Procrit (erythropoietin; Ortho Biotech), Epogen (erythropoietin; Amgen), Arnesp (erytropoietin). In addition to having specific or non-specific targets, immunotherapeutic agents can be active, i.e. stimulate the body's own immune response, or they can be passive, i.e. comprise immune system components that were generated external to the body. Passive specific immunotherapy typically involves the use of one or more monoclonal antibodies that are specific for a particular antigen found on the surface of a cancer cell or that are specific for a particular cell growth factor. Monoclonal antibodies may be used in the treatment of cancer in a number of ways, for example, to enhance a subject's immune response to a specific type of cancer, to interfere with the growth of cancer cells by targeting specific cell growth factors, such as those involved in angiogenesis, or by enhancing the delivery of other anticancer agents to cancer cells when linked or conjugated to agents such as chemotherapeutic agents, radioactive particles or toxins. Monoclonal antibodies currently used as cancer immunotherapeutic agents that are suitable for inclusion in the combinations of the present invention include, but are not limited to, rituximab (Rituxan®), trastuzumab (Herceptin®), ibritumomab tiuxetan (Zevalin®), tositumomab (Bexxar®), cetuximab (C-225, Erbitux®), bevacizumab (Avastin®), gemtuzumab ozogamicin (Mylotarg®), alemtuzumab (Campath®), and BL22. Other examples include anti-CTLA4 antibodies (e.g. Ipilimumab), anti-PD1 antibodies, anti-PDL1 antibodies, anti-TIMP3 antibodies, anti-LAG3 antibodies, anti-B7H3 antibodies, anti-B7H4 antibodies or anti-B7H6 antibodies. In some embodiments, antibodies include B cell depleting antibodies. Typical B cell depleting antibodies include but are not limited to anti-CD20 monoclonal antibodies [e.g. Rituximab (Roche), Ibritumomab tiuxetan (Bayer Schering), Tositumomab (GlaxoSmithKline), AME-133v (Applied Molecular Evolution), Ocrelizumab (Roche), Ofatumumab (HuMax-CD20, Gemnab), TRU-015 (Trubion) and IMMU-106 (Immunomedics)], an anti-CD22 antibody [e.g. Epratuzumab, Leonard et al., Clinical Cancer Research (Z004) 10: 53Z7-5334], anti-CD79a antibodies, anti-CD27 antibodies, or anti-CD19 antibodies (e.g. U.S. Pat. No. 7,109,304), anti-BAFF-R antibodies (e.g. Belimumab, GlaxoSmithKline), anti-APRIL antibodies (e.g. anti-human APRIL antibody, ProSci inc.), and anti-IL-6 antibodies [e.g. previously described by De Benedetti et al., J Immunol (2001) 166: 4334-4340 and by Suzuki et al., Europ J of Immunol (1992) 22 (8) 1989-1993, fully incorporated herein by reference]. The immunotherapeutic treatment may consist of allografting, in particular, allograft with hematopoietic stem cell HSC. The immunotherapeutic treatment may also consist in an adoptive immunotherapy as described by Nicholas P. Restifo, Mark E. Dudley and Steven A. Rosenberg “Adoptive immunotherapy for cancer: harnessing the T cell response, Nature Reviews Immunology, Volume 12, April 2012). In adoptive immunotherapy, the subject's circulating lymphocytes, NK cells, are isolated amplified in vitro and readministered to the subject. The activated lymphocytes or NK cells are most preferably be the subject's own cells that were earlier isolated from a blood or tumor sample and activated (or “expanded”) in vitro.

As used herein, the term “immune checkpoint inhibitor” refers to molecules that totally or partially reduce, inhibit, interfere with or modulate one or more immune checkpoint proteins.

As used herein, the term “immune checkpoint protein” has its general meaning in the art and refers to a molecule that is expressed by T cells in that either turn up a signal (stimulatory checkpoint molecules) or turn down a signal (inhibitory checkpoint molecules).

Examples of stimulatory checkpoint include CD27 CD28 CD40, CD122, CD137, OX40, GITR, and ICOS. Examples of inhibitory checkpoint molecules include A2AR, B7-H3, B7-H4, BTLA, CTLA-4, CD277, IDO, KIR, PD-1, PD-L1, LAG-3, TIM-3 and VISTA.

In some embodiments, the combination or composition of the present invention is administered with a radiotherapeutic agent. The term “radiotherapeutic agent” as used herein, is intended to refer to any radiotherapeutic agent known to one of skill in the art to be effective to treat or ameliorate cancer, without limitation. For instance, the radiotherapeutic agent can be an agent such as those administered in brachytherapy or radionuclide therapy. Such methods can optionally further comprise the administration of one or more additional cancer therapies, such as, but not limited to, chemotherapies, and/or another radiotherapy.

As used herein, the term “radiotherapy” for “radiation therapy” has its general meaning in the art and refers the treatment of cancer with ionizing radiation. Ionizing radiation deposits energy that injures or destroys cells in the area being treated (the target tissue) by damaging their genetic material, making it impossible for these cells to continue to grow. One type of radiation therapy commonly used involves photons, e.g. X-rays. Depending on the amount of energy they possess, the rays can be used to destroy cancer cells on the surface of or deeper in the body. The higher the energy of the x-ray beam, the deeper the x-rays can go into the target tissue. Linear accelerators and betatrons produce x-rays of increasingly greater energy. The use of machines to focus radiation (such as x-rays) on a cancer site is called external beam radiation therapy. Gamma rays are another form of photons used in radiation therapy. Gamma rays are produced spontaneously as certain elements (such as radium, uranium, and cobalt 60) release radiation as they decompose, or decay. In some embodiments, the radiation therapy is external radiation therapy. Examples of external radiation therapy include, but are not limited to, conventional external beam radiation therapy; three-dimensional conformal radiation therapy (3D-CRT), which delivers shaped beams to closely fit the shape of a tumor from different directions; intensity modulated radiation therapy (IMRT), e.g., helical tomotherapy, which shapes the radiation beams to closely fit the shape of a tumor and also alters the radiation dose according to the shape of the tumor; conformal proton beam radiation therapy; image-guided radiation therapy (IGRT), which combines scanning and radiation technologies to provide real time images of a tumor to guide the radiation treatment; intraoperative radiation therapy (IORT), which delivers radiation directly to a tumor during surgery; stereotactic radiosurgery, which delivers a large, precise radiation dose to a small tumor area in a single session; hyperfractionated radiation therapy, e.g., continuous hyperfractionated accelerated radiation therapy (CHART), in which more than one treatment (fraction) of radiation therapy are given to a subject per day; and hypofractionated radiation therapy, in which larger doses of radiation therapy per fraction is given but fewer fractions.

Further therapeutic active agent can be an antiemetic agent. Suitable antiemetic agents include, but are not limited to, metoclopramide, domperidone, prochlorperazine, promethazine, chlorpromazine, trimethobenzamide, ondansetron, granisetron, hydroxyzine, acetylleucine, alizapride, azasetron, benzquinamide, bietanautine, bromopride, buclizine, clebopride, cyclizine, dimenhydrinate, diphenidol, dolasetron, meclizine, methallatal, metopimazine, nabilone, pipamazine, scopolamine, sulpiride, tetrahydrocannabinols, thiethylperazine, thioproperazine and tropisetron. In a preferred embodiment, the antiemetic agent is granisetron or ondansetron.

In another embodiment, the further therapeutic active agent can be an hematopoietic colony stimulating factor. Suitable hematopoietic colony stimulating factors include, but are not limited to, filgrastim, sargramostim, molgramostim and epoietin alpha.

In still another embodiment, the other therapeutic active agent can be an opioid or non-opioid analgesic agent. Suitable opioid analgesic agents include, but are not limited to, morphine, heroin, hydromorphone, hydrocodone, oxymorphone, oxycodone, metopon, apomorphine, buprenorphine, meperidine, loperamide, ethoheptazine, betaprodine, diphenoxylate, fentanyl, sufentanil, alfentanil, remifentanil, levorphanol, dextromethorphan, phenazone, pemazocine, cyclazocine, methadone, isomethadone and propoxyphene. Suitable non-opioid analgesic agents include, but are not limited to, aspirin, celecoxib, rofecoxib, diclofenac, diflunisal, etodolac, fenoprofen, flurbiprofen, ibuprofen, ketoprofen, indomethacin, ketorolac, meclofenamate, mefenamic acid, nabumetone, naproxen, piroxicam and sulindac.

In yet another embodiment, the further therapeutic active agent can be an anxiolytic agent. Suitable anxiolytic agents include, but are not limited to, buspirone, and benzodiazepines such as diazepam, lorazepam, oxazepam, clorazepate, clonazepam, chlordiazepoxide and alprazolam.

In one embodiment, said additional active compounds may be contained in the same composition or administrated separately.

In another embodiment, the pharmaceutical combination or composition of the invention relates to combined preparation for simultaneous, separate or sequential use in the treatment of cancer in a subject in need thereof.

The invention also provides kits comprising the combination or composition of the invention. Kits containing the combination or composition of the invention find use in therapeutic methods.

The invention will be further illustrated by the following figures and examples. However, these examples and figures should not be interpreted in any way as limiting the scope of the present invention.

### FIGURES

FIG. 1. Dose-effect plots of FUra as a single agent [FUra], FUra with folinic acid [FUra-FA], FUra with PLP [FUra-PLP], and FUra with both folinic acid and PLP combined [FUra-FA-PLP] in the human colorectal carcinoma cell lines HT29 (A), and HCT116 (B), and in the murine lymphocytic leukemia L1210 (C).

FIG. 2. Combination indices (CI) with respect to fraction of cells inhibited (Fa) plots calculated for FUra with both folinic acid and PLP combined [FUra-FA-PLP] in the human colorectal carcinoma cell lines HT29 (A), and HCT116 (B), and in the murine lymphocytic leukemia L1210 (C). Dotted lines represent 95% confidence intervals.

FIG. 3. Mouse erythrocyte levels of PMP, PL, and PLP resulting from conversion of parenteral Pyridoxamine (PM) or pyridoxine (PN) given by the intraperitoneal route in high doses. Balb/C mice were given PM (A) or PN (B), either at 150 mg/kg or at 450 mg/kg at time 0 only, or twice at time 0 and after 12 hours from start. For each PM or PN dose explored, measurements were done after 1, 3, 6, 12, and 24 hours from start of the experiment.

### EXAMPLE

Fluorodeoxyuridine monophosphate (FdUMP), the active metabolite of 5-fluorouracil (FUra), binds to thymidylate synthase (TS) and CH₂—H₄PteGlu to form a ternary complex [FdUMP-TS-CH₂—H₄PteGlu] with concomitant inactivation of the TS (1-3) Stability of the complex increases as CH₂—H₄PteGlu level is augmented over a wide concentration range (2,3) Low concentrations of the cofactor lead to dissociation of the complex and recovery of the enzyme activity resulting in loss of cytotoxic potency of the fluoropyrimidines. Supplementation of cancer cell lines exposed to FUra or fluorodeoxyuridine (FUdR) with high concentration N5-formyl tetra hydro pteroylglutamate (5-HCO—H₄PteGlu; folinic acid; leucovorin) in vitro resulted in greater formation of ternary complex than with these fluoropyrimidines as single agents, leading to potentiation of the cytotoxic effect (4).

From these findings, investigators including ourselves designed regimens combining FUra and folinic acid in high doses for treatment of patients with various types of carcinomas and have demonstrated that the combination possess higher antitumor efficacy than that of FUra administered as a single agent (5-7). Based on these early studies, a number of schemas combining FUra and folinic acid became a basis for single-agent and combination therapies currently used for treatment of patients with colorectal, gastric and, more recently, pancreatic adenocarcinomas. Subsequent attempts were made with the aim to improve modulation of the fluoropyrimidines in the clinics by increasing the amounts of folinic acid, and by using the pure natural [6S]-stereoisomer ([6S]-5-HCO-tetrahydro pteroylglutamate] instead of the [6R,S]-mixture of enantiomers. However, at the present time there is no clear evidence of improvement of the anticancer potency of the fluoropyrimidines through these changes.

Effectiveness of the biochemical modulation of the fluoropyrimidines by folates upon cytotoxicity varies among cancer cells. Variation is thought to be due mainly to differences in intracellular expansion levels of CH₂—H₄PteGlu, and in polyglutamation capacities of cells, mainly for long chain length folylpolyglutamates (8).

Considering the effect of rising CH₂—H₄PteGlu concentration on the dissociation of FdUMP from the ternary complex (2,3), it is likely that neither supplementation of cancer cells with increasing doses of folate, nor supply with any folate compound, would result in high intracellular levels of CH₂—H₄PteGlu for times required for optimal TS inhibition, as suggested from previous experiments.8-15 In the majority of these studies, when cancer cells were exposed to great amounts of folates only small expansion of CH₂—H₄PteGlu was observed, whose concentration rapidly decreased after discontinuation of folate exposure (8-15).

One possible explanation for these findings is the very rapid turnover of folates in cancer cells (16) resulting from interconversion of folate cofactors including the irreversible reduction of CH₂—H₄PteGlu to N5-methyl tetra hydro pteroylglutamate (CH₃—H₄PteGlu), that is the most abundant intracellular folate (15,16). Consequently, mobilization of CH₃—H₄PteGlu to enter the folate interconvertible pool is dependent on formation of H₄PteGlu resulting from the methionine synthase-catalyzed synthesis of L-methionine from L-homocysteine.

Poor expansion of the intracellular CH₂—H₄PteGlu pools may also result from low production of CH₂—H₄PteGlu in cancer cells. Synthesis of CH₂—H₄PteGlu from H₄PteGlu results from two metabolic pathways. One is catalysed by serine hydroxymethyl transferase (SHMT), a ubiquitous PLP-dependent enzyme which consists in two isoforms, the cytoplasmic SHMT isoform 1, and the mitochondrial SHMT isoform 2 (17,18). The SHMT catalyzes the reversible transfer of Cβ of serine to H₄PteGlu, with formation of glycine and CH₂—H₄PteGlu. This reaction is a primary source of one carbon units required for reactions catalyzed by folate-dependent enzymes, including synthesis of thymidylate (dTMP) from deoxyuridylate (dUMP) catalysed by TS, the target enzyme of FdUMP.

The second pathway is the Glycine Cleavage System that catalyzes glycine cleavage up to formation of CH₂—H₄PteGlu in three successive reactions comprising three enzymes and a carrier protein bound to the mitochondrial inner membrane (18,19). The carrier is H-protein, and enzymes are P-protein, a PLP-dependent glycine dehydrogenase; T-protein, an amino methyltransferase; and L-protein, a dihydro lipoamide dehydrogenase.

The rationale for the present hypothesis lies in the affinity between SHMT apoenzyme and cofactor. The dissociation constant (Kd) of SHMT for PLP was measured from various animal and human enzyme sources. In one early work, PLP was found to bind purified bovine liver cSHMT with a Kd as high as 27 μmon (20), but smaller values were found by other investigators. In one study, cofactor bound to purified rabbit liver SHMT with a Kd of 700 nmol/L (21). Human recombinant cytoplasmic cSHMT bound to cofactor with a Kd of 850 nmol/L in one study (22), and in another study human recombinant SHMT1 and SHMT2 bound to cofactor with Kds of 250 nmol/L, and 440 nmol/L, respectively (23). By contrast, levels of naturally occurring PLP in human erythrocytes under basal conditions were reported as small as 30-100 nmol/L of packed cells (24), which strikingly differs from reported binding affinities of SHMT apoenzyme for cofactor. Although little is known of the mechanism by which intracellular cofactor is supplied to the PLP-dependent enzymes (25), these features predict that SHMT activity should be sensitive to PLP concentration changes within cells. Vitamin B6 deficiency in rat produces loss of SHMT activity in liver, and impairment of one-carbon metabolism (26). Furthermore, vitamin B6 restriction has been reported to decrease the activity of SHMT in MCF-7 human mammary carcinoma cells in vitro which suggests that availability of adequate amounts of the cofactor is needed for increasing enzyme function within cancer cells (22).

We hypothesized that supplementation of cancer cells with high amounts of PLP would facilitate production of CH₂—H₄PteGlu and modulate FUra by augmenting its cytotoxic activity through [TS-CH₂—H₄PteGlu-FdUMP] ternary complex stabilization. To test the hypothesis, experiments were conducted in three cancer cell line models in vitro to assess for interactions between FUra, folinic acid and PLP in high concentration upon cytotoxicity.

Material & Methods

Cell Lines and Cytotoxicity Studies In Vitro

The human colorectal carcinoma cell lines HT29, and HCT116 and the murine L1210 lymphocytic leukemia were thawed from mycoplasma-free frozen stocks and were controlled for contamination. Cells were grown in customized DMEM cell culture medium without any B6-vitamer (Gibco; Life Technologies) supplemented with 10% FBS and antibiotics (streptomycin, 50 μg/ml, and penicillin, 50 U/ml) at 37° C. in an atmosphere containing 5% CO2.

Cancer cells were exposed in 12 well-cell plates to FUra in various concentrations under four conditions; either as a single agent [FUra]; in combination with [6R,S]-folinic acid (20 μmon) [FUra-FA]; in combination with PLP (160 μmon; Sigma-Aldrich) [FUra-PLP]; and with both agents [6R,S]-folinic acid (20 μmon), and PLP (160 μmon) together [FUra-AF-PLP]. Cells were harvested 72 hours from start of the exposure. Cell viability was measured with the Trypan Blue dye exclusion test in Malassez chambers or by flow cytometry. For the latter method, living cells defined by light double scatter were counted in a BD Accuri C6 flow cytometer (BD Biosciences). Experiments were in duplicate.

We have analyzed cell growth of the HT29 cells in customized medium without any B6-vitamer and did not observe any impact on growth after as much as five successive passages of 96-hour cultures. We have also demonstrated that neither folinic acid nor PLP carry cytotoxic properties per se in concentrations used in the present experiment.

Growth inhibition data obtained with FUra as a single agent [FUra], FUra with folinic acid [FUra-FA], FUra with PLP [FUra-PLP], and FUra with both folinic acid and PLP combined [FUra-FA-PLP] were studied according to the Median Effect principle for concentration-effect analysis. The combination index (CI) proposed by Chou and Talalay considering two mutually non-exclusive drugs (i.e., [FUra-FA] was Drug 1, and [FUra-PLP] was Drug 2, based on the assumption that the bio modulators act on exclusive targets) was used for determination of synergism, summation or antagonism.27 Combination indices were calculated from the effect on cell growth produced by the combinations of [FUra-FA-PLP], [FUra-FA], and [FUra-PLP] at 1:1 constant FUra concentration ratio. For representation of synergism (CI<1), summation (CI≈1) and antagonism (CI>1), the combination indices for [FUra-FA-PLP] were plotted as CI with respect to percent of cells inhibited (Fraction affected; Fa). Dose-effect and CI parameters were calculated, and dose-effect and Fa-CI plots were done with the CalcuSyn software (Biosoft, Cambridge, UK).

Intracellular Conversion of B6 Vitamers In Vivo

Erythrocyte pharmacokinetics of B6 vitamers was studied in mice after parenteral pyridoxamine (PM) or Pyridoxine (PN) in high doses with the aim at exploring the physiologic limits of cells to accumulate PLP in vivo.

Vitamin B6 is the generic name that encompasses six inter convertible compounds (i.e., B6 vitamers), namely pyridoxine (PN), pyridoxamine (PM), pyridoxal (PL), and their respective 5′-phosphorylated forms, PNP, PMP and the cofactor PLP.

We measured mouse erythrocyte levels of PMP, PL, and PLP resulting from conversion of parenteral PM or Pyridoxine (PN) given in high doses. Female Balb/C mice aged 6 weeks, caged and fed under standard conditions were given intraperitoneal PM (Sigma-Aldrich) either at 150 mg/kg or at 450 mg/kg at time 0 only (t0), or twice at time 0 and after 12 hours from start (i.e., at times t0 and at t12 h). For each PM dose explored, groups of two mice each subjected to one injection of PM at t0 were sacrificed and sampled after 1, 3, 6, and 12 hours from start, and two animals that received 2 injections of PM were sampled 12 hours after the 2nd injection (i.e., 24 hours after start of the experiment). Blood was collected in heparin. Measurements of B6 vitamers were done by HPLC.28

Results

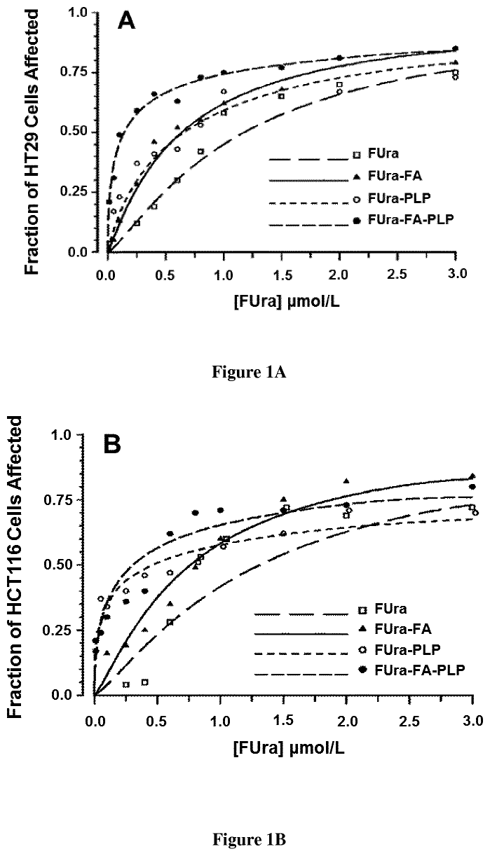

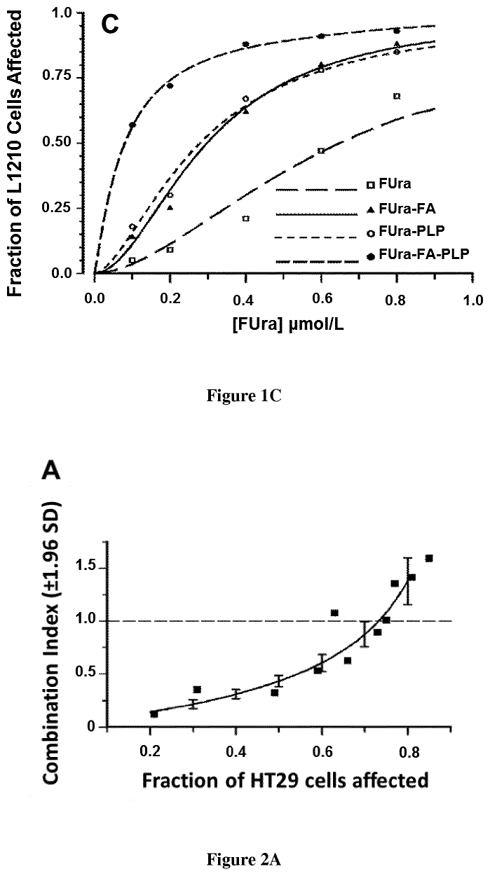

Dose-effect plots are represented in FIGS. 1A, 2A and 3A, and the 50% inhibitory concentrations (IC50s) in each experimental condition are shown in Table 1. Results demonstrate that, in the three cell lines studied, FUra cytotoxicity was increased by FA, as well as by PLP. Cytotoxicity was greater with FUra combined with both FA and PLP together.

TABLE 1
Changes in 50%-inhibitory concentrations (IC50s) of FUra in the human
colorectal carcinoma cell lines HT29 and HCT116, and in the L1210
murine lymphocytic leukemia after 72 hour-incubation in the presence
of Folinic Acid (FA, 20 μmol/L), Pyridoxal 5′-Phosphate (PLP,
160 μmol/L), and both FA (20 μmol/L), and PLP (160 μmol/L)
combined.

| | | IC₅₀ in μmol/L of |
|---|---|---|
| Cell Line | Drugs | FUra (95% CI) |
| HT29 | FUra | 1.18 (0.76-1.82) |
| carcinoma | FUra-FA | 0.64 (0.56-0.72) |
| | FUra-PLP | 0.66 (0.47-0.91) |
| | FUra-FA-PLP | 0.14 (0.12-0.17) |
| HCT116 | FUra | 1.31 (0.74-2.32) |
| carcinoma | FUra-FA | 0.76 (0.56-1.04) |
| | FUra-PLP | 0.46 (0.34-0.62) |
| | FUra-FA-PLP | 0.31 (0.21-0.45) |
| L1210 | FUra | 0.47 (0.32-0.69) |
| leukaemia | FUra-FA | 0.22 (0.18-0.26) |
| | FUra-PLP | 0.37 (0.34-0.40) |
| | FUra-FA-PLP | 0.10 (0.08-0.12) |

Dose-effect parameters, IC₅₀s and 95% confidence intervals were obtained with the CalcuSyn Software (Biosoft).

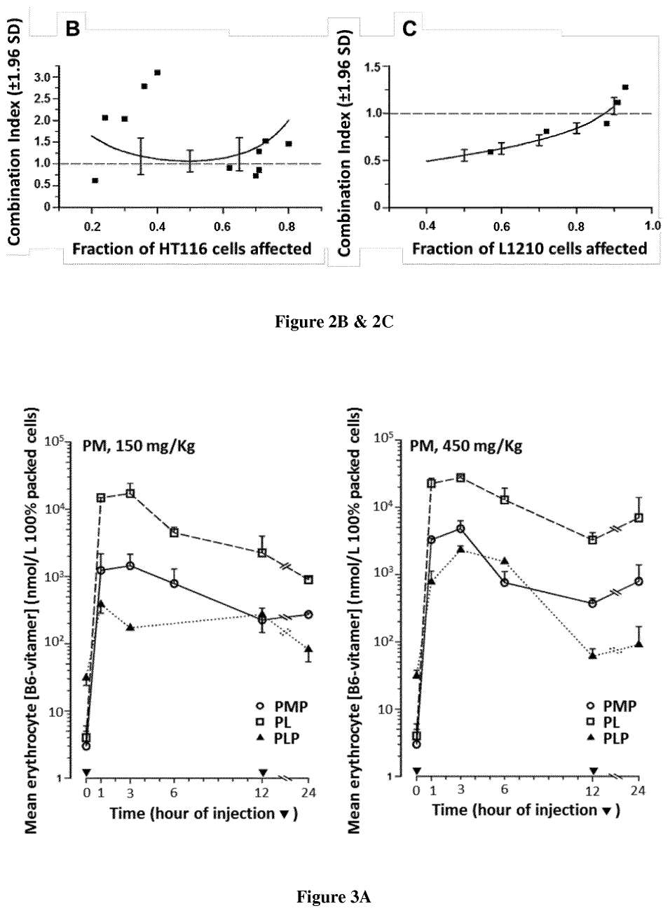

Combination Indices for [FUra-FA-PLP] were of synergistic (<1) and additive (≈1) significance for most experimental values obtained for fractions of affected cells (Fa) below ≤75% in HT29 and L1210 cells (FIGS. 2A, and 2C). Within these fractional effect limits, CI simulations followed a continuous trend for synergy of the effect of FA and PLP combined upon FUra cytotoxicity. For HCT116 cells, experimental CI values are more dispersed than that for HT29 and L1210 cells (FIG. 2B). A number of values obtained for fractions of HCT116 affected cells between 20% and 70% are of synergistic significance, while others are >1, mostly within fractional effect extremes; in this cell line, CI simulations follow a trend for additivity of the effect of FA and PLP combined on FUra cytotoxicity within fractional effect limits of approximately 30%-70%.

Erythrocytes Rapidly Metabolize Pyridoxamine and Accumulate High Concentrations of PLP

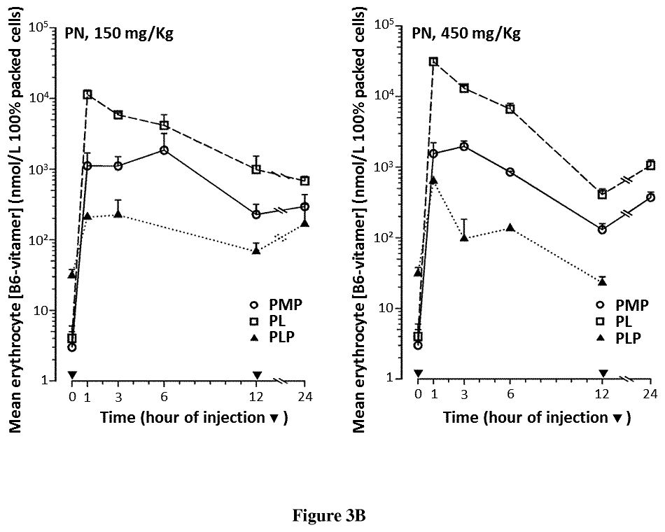

Basal PLP erythrocyte concentration was 31±7 nmol/L of 100% packed red cells (FIG. 3). Parenteral PM and PN resulted in expansion of intracellular PLP pools. Conversion into PL and PMP are approximately similar in mice given with each of the two vitamers. However, at the highest dose of B6 vitamer, PLP expansion was higher with PM than with PN. Precise comparisons between the efficacy of these compounds in terms of PLP conversion cannot be done at present, and further studies are necessary. PLP levels rapidly decreased after the third hour, approaching baseline concentrations by 12 hours after injection. This observation was similar in animals that received two injections of PM or PN (i.e., at times t0 and t12 h), indicating that cellular clearance of newly synthesized PLP is rapid and that cofactor is not appreciably accumulated in cells when injections are repeated at 12-hour interval. (FIGS. 3A and 3B). Metabolic conversion of PM also resulted in production of large amounts of PL, and PMP. Peak PLP concentrations were about 6-fold greater in erythrocytes from animals that have been injected with the highest dose of vitamin B6 (i.e., 450 mg/kg of PM) than in those that received 150 mg/kg; mean peak PLP levels were 382 nmol/L of 100% packed cells in mice that received 150 mg/kg PM, and 2,326 nmol/L of 100% packed cells in mice that received PM at 450 mg/kg (FIG. 3A).

No signs of acute toxicity were observed in any mice receiving either one or two injections of PM at any of the two doses studied.

Discussion

Dose-effect data confirm modulation of FUra by high concentration folinic acid, resulting in potentiation of the cytotoxic effect (4). Results suggest that FUra combined with high-concentration PLP follow a trend towards potentiation upon cytotoxicity. Combination of FUra, folinic acid, and PLP resulted in greater cytotoxic effect in the three cell lines studied. Combination indices indicate that FA and PLP together add up their effects or exert a synergistic interaction with FUra upon cytotoxicity for most experimental data obtained in HT29, L1210 cells and, with lesser magnitude, in HCT116 cells.

Although the present results fit our assumption, determination of the precise mechanism of the interaction is essential and demands further research. From our data, we hypothesize that vitamin B6 facilitates expansion of CH₂—H₄PteGlu in cancer cells leading to increased ternary complex stabilization in the presence of FUra.

Effective uptake of PLP by cells in vitro was previously demonstrated in erythrocytes (29,30). However, neither intracellular concentration levels attained after incubation with high concentration PLP, nor metabolism of incorporated cofactor have been studied under these conditions.

The present study demonstrates that intracellular levels of PLP are strongly augmented after parenteral administration of vitamin B6 in high doses. With these doses, PLP attains levels close or greater than the Kd reported for binding of cofactor to SHMT, which anticipates that activity of the enzyme should be improved by intracellular PLP pool expansion in vivo. However, decline of free intracellular PLP to basal levels rapidly occurs in less than 12 hours from injection. The mechanisms underlying this feature have not been elucidated. Binding of PLP to haemoglobin is one possible cause. Regulatory mechanisms to avoid PLP accumulation in high concentration may exist as well (18). Our findings are in accordance with that reported on intracellular pharmacokinetics of PMP, PL, and PLP after parenteral administration of PN in humans (18,24).

The present findings should be considered for experiments aiming at improving bio modulation of the fluoropyrimidines by folinic acid and B6 vitamers in tandem for treatment of cancer.

### REFERENCES

Throughout this application, various references describe the state of the art to which this invention pertains. The disclosures of these references are hereby incorporated by reference into the present disclosure.
1. Santi D V, McHenry C S, Sommer H. Mechanism of interaction of thymidylate synthetase with 5-fluorodeoxyuridylate. Biochemistry 1974. 13: 471-81
2. Danenberg P V, Danenberg K D. Effect of 5,10-methylenetetrahydrofolate on the dissociation of 5-Fluoro-2′-deoxyuridylate from thymidylate synthetase: evidence for an ordered mechanism. Biochemistry 1978. 17: 4018-24
3. Lockshin A, Danenberg P V. Biochemical factors affecting the tightness of fluorodeoxyuridylate binding to human thymidylate synthetase. Biochemical Pharmacology 1981.30: 247-57
4. Ullman B, Lee M, Martin D W and Santi D V, Cytotoxicity of 5-fluoro-2′-deoxyuridine: Requirement for reduced folate cofactors and antagonism by methotrexate. Proc Natl Acad Sci USA 1978. 75: 980-3
5. Machover D, Schwarzenberg L, Goldschmidt E, Tourani J-M, Michalski B, Hayat M, Dorval T, Misset J-L, Jasmin C, Maral J, Mahe G. Treatment of advanced colorectal and gastric carcinomas with 5-fluorouracil combined with high-dose folinic acid. A pilot study. Cancer Treat Rep 66: 1803-7, 1982.
6. Machover D, Goldschmidt E, Chollet P, Metzger G, Zittoun J, Marquet J, Vandenbulcke J M, Misset J L, Schwarzenberg L, Fourtillan J B, Gaget H, Mahe G. Treatment of advanced colorectal and gastric adenocarcinomas with 5-fluorouracil and high-dose folinic acid. Journal of Clinical Oncology 1986 4: 685-696.
7. Piedbois P, Buyse M, Rustum Y, Machover D, Erlichman C, Carlson R W, Valone F, Labianca R, Doroshow J H, Petrelli N for the Advanced Colorectal Cancer Meta-Analysis Project. Modulation of 5-fluorouracil by leucovorin in patients with advanced colorectal cancer: Evidence in terms of response rate. Journal of Clinical Oncology 1992. 10: 896-903
8. Romanini A, Lin J T, Niedzwiecki D, Bunni M, Priest D G, Bertino J R. Role of folylpolyglutamates in biochemical modulation of fluoropyrimidines by leucovorin. Cancer Res 1991. 51: 789-93
9. Houghton J A, Williams L G, Cheshire P J, Wainer I W, Jadaud P, Houghton P J. Influence of dose of [6RS]-leucovorin on reduced folate pools and 5-fluorouracil-mediated thymidylate synthase inhibition in human colon adenocarcinoma xenografts. Cancer Res 1990. 50: 3940-6
10. Priest D G, Schmitz J C, Bunni M A. Folate metabolites as modulators of antitumor drug activity. In: Chemistry and Biology of Pteridines and Folates (Ed. Ayling J E), pp. 693-697. Plenum Press, New York, 1993.
11. Voeller D M, Allegra C J. Intracellular metabolism of 5-methyltetrahydrofolate and 5-formyltetrahydrofolate in a human breast cancer cell line. Cancer Chemother Pharmacol 1994. 34: 491-6
12. Wright J E, Dreyfuss A, El-Magharbel I, Trites D, Jones S M, Holden S A, Rosowsky A, Frei III E. Selective expansion of 5,10-Methylenetetrahydrofolate pools and modulation of 5-fluorouracil antitumor activity by leucovorin in vivo. Cancer Res 1989. 49: 2592-6
13. Boarman D M, Allegra C J. Intracellular metabolism of 5-formyl tetrahydrofolate in human breast and colon cell lines. Cancer Res 1992. 52: 36-44
14. Zhang Z-G, Rustum Y M. Effect of diastereoisomers of 5-formyltetrahydrofolate on cellular growth, sensitivity to 5-fluoro-2′-deoxyuridine, and methylenetetrahydrofolate polyglutamate levels in HCT-8 cells. Cancer Res 1991. 51: 3476-81
15. Machover D, Zittoun J, Broët P, Metzger G, Orrico M, Goldschmidt E, Schilf A, Tonetti C, Tan Y, Delmas-Marsalet B, Luccioni C, Falissard B, Hoffman R. Cytotoxic synergism of methioninase in combination with 5-fluorouracil and folinic acid. Biochemical Pharmacology 2001. 61: 867-76
16. Nixon P F, Slutsky G, Nahas A and Bertino J R. The turnover of folate coenzymes in murine lymphoma cells. J Biol Chem 1973. 248: 5932-6
17. Florio R, di Salvo M L, Vivoli M, Contestabile R. Serine hydroxymethyltransferase: A model enzyme for mechanistic, structural, and evolutionary studies. Biochimica et Biophysica Acta 2011. 1814: 1489-96
18. Ueland P M, Ulvik A, Rios-Ávila L, Midttun Ø, Gregory J F. Direct and functional biomarkers of Vitamin B6 status. Annu Rev Nutr 2015. 35: 33-70
19. Kikuchi G, Motokawa Y, Yoshida T, Hiraga K. Glycine cleavage system: reaction mechanism, physiological significance, and hyperglycinemia. Proc Jpn Acad Ser B Phys Biol Sci 2008. 84: 246-63
20. Jones III C W, Priest D G. Interaction of pyridoxal 5′-phosphate with apo-serine hydroxymethyl transferase. Biochimica et Biophysica Acta 1978. 526: 369-74
21. Schirch L V, Edmiston M, Chen M S, Barra D, Bossa F, Hinds L, Fasella P. Serine transhydroxymethylase. Subunit structure and the involvement of sulfhydryl groups in the activity of the enzyme. J Biol Chem 1973. 248: 6456-61. (Quoted by Perry et al. in reference 22)
22. Perry C, Yu S, Chen J, Matharu K S, Stover P J. Effect of vitamin B6 availability on serine hydroxymethyltransferase in MCF-7 cells. Archives of Biochemistry and Biophysics 2007. 462: 21-7
23. Giardina G, Brunotti P, Fiascarelli A, Cicalini A, Costa MGS, Buckle A M, di Salvo M L, Giorgi A, Marani M, Paone A, Rinaldo S, Paiardini A, Contestabile R, Cutruzzola F. How pyridoxal 5′-phosphate differentially regulates human cytosolic and mitochondrial serine hydroxymethyltransferase oligomeric state. FEBS Journal 2015. 282: 1225-41
24. Zempleni J, Kübler W. The utilization of intravenously infused pyridoxine in humans. Clinica Chimica Acta 1994. 229: 27-36
25. Di Salvo M L, Contestabile R, Safo M K. Vitamin B6 salvage enzymes: mechanism, structure and regulation. Biochimica et Biophysica Acta 2011. 1814: 1597-608
26. Martinez M, Cuskelly G J, Williamson J, Toth J P, Gregory III J F. Vitamin B-6 deficiency in rats reduces hepatic serine hydroxymethyl transferase and cystathionine β-synthase activities and rates of in vivo protein turnover, homocysteine remethylation and transsulfuration. J Nutr 2000, 130: 1115-23
27. Chou T C, Talalay P. Quantitative analysis of dose-effect relationships: the combined effects of multiple drugs or enzyme inhibitors. Adv Enzyme Regul 1984. 22: 27-55
28. Bisp M R, Bor M V, Heinsvig E M, Kall M A, and Naxø E. Determination of B6 vitamers and pyridoxic acid in plasma: development and evaluation of a high-performance liquid chromatography assay. Anal Biochem 2002. 305: 82-9
29. Suzue R, Tachibana M. The uptake of pyridoxal phosphate by human red blood cells. The Journal of Vitaminology 1970. 16: 164-71
30. Maeda N, Takahashi K, Aono K, Shiga T. Effect of pyridoxal 5′-phosphate on the oxygen affinity of human erythrocytes. British Journal of Haematology 1976. 34: 501-9.

1-15. (canceled)

16. An antitumor pharmaceutical composition for treating cancer in a subject in need thereof comprising (i) a fluoropyrimidine, (ii) a B6 vitamer, and (iii) a folate

17. The antitumor pharmaceutical composition according to claim 16 for simultaneous, separate or sequential administration for the treatment of cancer in a subject in need thereof.

18. The antitumor pharmaceutical composition according to claim 16, wherein the fluoropyrimidine is selected from the group consisting of 5-fluorouracil, capecitabine, 5-fluoro-2′-deoxyuridine, ftorafur, emitefur, eniluracil/5-FU, S-1, UFT and mixtures thereof.

19. The antitumor pharmaceutical composition according to claim 18, wherein the fluoropyrimidine is 5-fluorouracil.

20. The antitumor pharmaceutical composition according to claim 16, wherein the B6 vitamer is selected from the group consisting of pyridoxine, pyridoxal, pyridoxamine, 5′-phosphorylated derivatives thereof, acetate esters thereof, pharmaceutically compatible salts thereof, and mixtures thereof.

21. The antitumor pharmaceutical composition according to claim 20, wherein the B6 vitamer is selected from the group consisting of pyridoxine and pyridoxamine.

22. The antitumor pharmaceutical composition according to claim 16, wherein the B6 vitamer is administered at a high dose enabling to achieve plasma and/or intracellular levels of PLP equal or greater than that required for optimum synergistic effect of the fluoropyrimidines.

23. The antitumor pharmaceutical composition according to claim 16, wherein the folate is selected from the group consisting of folic acid, dihydrofolate, tetrahydrofolate, 5-methyltetrahydrofolate, 5,10-methylenetetrahydrofolate, 5,10-methenyltetrahydrofolate, 5-formiminotetrahydrofolate, 5-formyltetrahydrofolate, [6S]-5-formyltetrahydrofolate, 10-formyltetrahydrofolate, pharmaceutically compatible salts thereof, and mixtures thereof.

24. The antitumor pharmaceutical composition according to claim 23, wherein the folate is 5-formyltetrahydrofolate or [6S]-5-formyltetrahydrofolate.

25. The antitumor pharmaceutical composition according to claim 16, wherein said composition is administered sequentially or concomitantly with one or more immunotherapeutic, chemotherapeutic or radiotherapeutic agent.

26. The antitumor pharmaceutical composition for treating cancer in a subject in need thereof according to claim 16 wherein said composition is a combined preparation comprising (i) one or more dosage units of a fluoropyrimidine, (ii) one or more dosage units of a B6 vitamer, and (iii) one or more dosage units of a folate.

27. A kit for treating cancer in a subject in need thereof comprising:
a) (i) an antitumor pharmaceutical composition comprising a fluoropyrimidine and a B6 vitamer and (ii) one or more dosage units of a folate, or
b) (i) an antitumor pharmaceutical composition comprising a fluoropyrimidine and (ii) one or more dosage units of a pharmaceutical composition comprising a B6 vitamer and a folate, or
c) (i) an antitumor pharmaceutical composition comprising a fluoropyrimidine, (ii) one or more dosage units of a B6 vitamer and (iii) one or more dosage units of a folate.

28. A method for treating cancer comprising the step of administering to a subject in need thereof a therapeutically effective amount of an antitumor pharmaceutical composition comprising (i) a fluoropyrimidine, (ii) a B6 vitamer and (iii) a folate

29. The method for treating cancer according to claim 28, wherein an antitumor pharmaceutical composition comprising (i) a fluoropyrimidine, (ii) a B6 vitamer and (iii) a folate is administered simultaneously, separately or sequentially to a subject in need thereof

30. The method according to claim 29, wherein the fluoropyrimidine is selected from the group consisting of 5-fluorouracil, capecitabine, 5-fluoro-2′-deoxyuridine, ftorafur, emitefur, eniluracil/5-FU, S-1, UFT and mixtures thereof.

31. The method according to claim 28, wherein the B6 vitamer is selected from the group consisting of pyridoxine, pyridoxal, pyridoxamine, 5′-phosphorylated derivatives thereof, acetate esters thereof, pharmaceutically compatible salts thereof, and mixtures thereof.

32. The method according to claim 31 wherein the B6 vitamer is administered at a high dose enabling to achieve plasma and/or intracellular levels of PLP equal or greater than that required for optimum synergistic effect of the fluoropyrimidines.

33. The method according to claim 28 wherein the folate is selected from the group consisting of folic acid, dihydrofolate, tetrahydrofolate, 5-methyltetrahydrofolate, 5,10-methylenetetrahydrofolate, 5,10-methenyltetrahydrofolate, 5-formiminotetrahydrofolate, 5-formyltetrahydrofolate, [6S]-5-formyltetrahydrofolate, 10-formyltetrahydrofolate, pharmaceutically compatible salts thereof, and mixtures thereof.

34. The method according to claim 28, wherein a therapeutically effective amount of an antitumor pharmaceutical composition comprising (i) a fluoropyrimidine, (ii) a B6 vitamer and (iii) a folate is administered sequentially or concomitantly with one or more immunotherapeutic, chemotherapeutic or radiotherapeutic agent.

35. The method according to claim 28 for treating cancer comprising the step of administering to a subject in need thereof a therapeutically effective amount of an antitumor pharmaceutical composition comprising (i) a fluoropyrimidine which is 5-fluorouracil (ii) a B6 vitamer which is selected from the group consisting of pyridoxine and pyridoxamine and (iii) a folate which is [6S]-5-formyltetrahydrofolate.
